(19) United States
(12) Patent Application Publication
(10) Pub. No.: US 2019/0254641 A1
(43) Pub. Date: Aug. 22, 2019

(54) BIOFLUID SENSING DEVICES WITH SENSOR ABRASION PROTECTION

(71) Applicant: Eccrine Systems, Inc., Cincinnati, OH (US)

(72) Inventors: Gavi Begtrup, Cincinnati, OH (US); Jason Heikenfeld, Cincinnati, OH (US); Austin Morgan, Bellevue, KY (US)

(73) Assignee: Eccrine Systems, Inc., Cincinnati, OH (US)

(21) Appl. No.: 16/403,094

(22) Filed: May 3, 2019

Related U.S. Application Data
(63) Continuation-in-part of application No. 15/382,703, filed on Dec. 18, 2016.
(60) Provisional application No. 62/666,921, filed on May 4, 2018.
(60) Provisional application No. 62/269,254, filed on Dec. 18, 2015.

(51) Int. Cl. A61B 10/00; A61B 5/145; A61B 5/00

(52) U.S. Cl. CPC A61B 10/0064; A61B 5/6833; A61B 5/14521; A61B 5/14546

(57) ABSTRACT

The disclosed invention provides a biofluid sensing device configured with a membrane-enhanced sensor located in a sweat collector. The disclosed analyte-specific sensor is configured to reduce required biofluid sample volume due to its close proximity to the skin and source of sweat biofluid. The sensor is contained within a pH and salinity-stabilized fluid that, in turn, is contained in a selectively permeable membrane to improve sensor performance in variable biofluids, and to protect the sensor from skin contact. In one embodiment, the biofluid sensing device includes means to protect the self-aligning sensors from abrasion against the skin or device components. Other embodiments of the disclosed invention include a track-etched membrane to provide sensor protection.

### CROSS-REFERENCE TO RELATED APPLICATIONS

This application is a continuation-in-part of U.S. application Ser. No. 15/382,703, filed Dec. 18, 2016, and claims priority to U.S. Provisional Application No. 62/269,254, filed Dec. 18, 2015 and U.S. Provisional Application No. 62/666,921, filed May 4, 2018, and has specification that relates to PCT/US16/43771, filed Jul. 23, 2016, the disclosures of which are hereby incorporated by reference herein in their entirety.

### BACKGROUND OF THE INVENTION

Wearable biofluid sensing technologies have enormous potential for applications ranging from athletics, to neonatology, to pharmacological monitoring, to personal digital health, to name a few applications. Sweat contains many of the same biomarkers, chemicals, or solutes that are carried in blood and can provide significant information enabling one to diagnose illness, health status, exposure to toxins, performance, and other physiological attributes even in advance of any physical sign. Furthermore, sweat itself, the action of sweating, and other parameters, attributes, solutes, or features on, near, or beneath the skin can be measured to further reveal physiological information.

Of all the other physiological fluids used for bio monitoring (e.g., blood, urine, saliva, tears), sweat has arguably the least predictable sampling rate in the absence of technology. However, with proper application of technology, sweat can be made to outperform other non-invasive or less invasive biofluids in predictable sampling. For example, it is difficult to control saliva or tear rate without negative consequences for the user (e.g., dry eyes, tears, dry mouth, or excessive saliva while talking). Urine is also a difficult fluid for physiological monitoring, because it is inconvenient to take multiple urine samples, it is not always possible to take a urine sample when needed, and control of biomarker dilution in urine imposes further significant inconveniences on the user or test subject.

Known and existing methods of reducing sweat volume and increasing sampling rate predictability include those reported frequently in the clinical literature, such as coating the skin with petroleum jelly or oil through which sweat can push. However, these techniques have been demonstrated only for sweat collection and are not necessarily compatible with a wearable sensor. For example, petroleum jelly would wet against the sensor and effectively seal it from any sweat. Furthermore, other possible sweat pressure-activated methods must somehow be affixed to skin so that sweat is confined horizontally (otherwise sweat pressure activation is not possible). Conventional approaches will not work with wearable sensors, and inventive steps are required for enablement. Clearly, the state of art is lacking in devices to properly reduce the volume between sensors and skin, which is critical for fast sampling times or for sampling during intervals with very low sweat rates. In addition, it also may be critical for prolonged stimulation (i.e., where less stimulation is required over longer periods), and for improving biomarker measurements where a low sweat rate is required to ensure correlation between biomarker concentrations in sweat and those in blood.

One novel method of reducing sweat volume as disclosed in PCT/US2016/043771 involves using pressure-activated sealants to horizontally confine sweat flow and reduce sweat volume. In order to reduce sweat volume, however, sweat pressure-activated methods also require the sensor to be properly aligned with sweat glands, which can prove difficult. Since it would be impractical for sweat sensing device users to reliably place a device in ideal alignment with sweat glands, devices may be designed to optimize sweat gland coverage when the device is randomly placed on skin. However, even with such designs, sweat gland density may vary with between individuals, or even body location on the same individual. Therefore, a sweat sensing device that is self-aligning with sweat glands may improve sensor proximity to sweat glands under a variety of circumstances, thereby reducing sweat volume.

However, self-aligning sweat sensing designs must also be configured to access prolonged sweat stimulation, which is a significant challenge. Further, as with other referenced means of reducing sweat volume, self-aligning sensors must also be protected from abrasion. The disclosed invention, therefore, discloses a means of providing prolonged sweat stimulation for abrasion-protected self-aligning sensors by configuring a sweat-stimulating chemical in close proximity to the sensors, and enabling sudomotor axon reflex sweat response through diffusion of the sweat stimulation compound into the skin.

### SUMMARY OF THE INVENTION

The disclosed invention provides a biofluid sensing device configured with a membrane-enhanced sensor located in a sweat collector. The disclosed analyte-specific sensor is configured to reduce biofluid sample volume due to its close proximity to the skin and source of sweat biofluid. The sensor is contained within a pH and salinity-stabilized fluid that, in turn, is contained in a selectively permeable membrane to improve sensor performance in variable biofluids, and to protect the sensor from skin contact. In one embodiment, the biofluid sensing device includes means to protect the self-aligning sensors from abrasion against the skin or device components. Other embodiments of the disclosed invention include a track-etched membrane to provide sensor protection.

### BRIEF DESCRIPTION OF THE DRAWINGS

The objects and advantages of the disclosed invention will be further appreciated in light of the following detailed descriptions and drawings in which:

FIG. 1 depicts at least a portion of a wearable device for sweat biosensing.

FIG. 2 depicts at least a portion of a wearable device for sweat biosensing.

FIG. 3 depicts at least a portion of a wearable device for sweat biosensing.

FIG. 4 depicts at least a portion of a wearable device for sweat biosensing.

FIG. 5 is a cross-sectional view of at least a portion of a wearable device for sweat biosensing with sensors and sweat stimulation means that self-align with sweat ducts.

FIG. 6 is a cross-sectional view of at least a portion of a wearable device for sweat biosensing with sensors and sweat stimulation means that self-align with sweat ducts.

FIG. 7 is a cross-sectional view of at least a portion of a wearable device for sweat biosensing with sensors and sweat stimulation means that self-align with sweat ducts.

FIG. 7A is a top view of at least a portion of a wearable device for sweat biosensing with sensors and sweat stimulation means that self-align with sweat ducts.

FIG. 8A is a cross-sectional view of at least a portion of a wearable device for biofluid sensing showing a membrane-enhanced sensor inside of a sweat collector.

FIG. 8B is a more detailed, cross-sectional view of the membrane-enhanced sensor of FIG. 8A.

FIG. 8C is a detailed, cross-sectional view of the sensor of FIG. 8A, showing the addition of a sweat stimulating component to the membrane-enhanced sensor.

FIG. 9 is a cross-sectional view of at least a portion of a wearable device for biofluid sensing showing a sensor and sweat stimulation components inside a collector.

FIG. 10A is a cross-sectional view of at least a portion of a wearable device for biofluid sensing having a sensor on the surface of a sweat collector.

FIG. 10B is a more detailed, cross-sectional view of the membrane-enhanced sensor of FIG. 10A.

FIG. 11A is a top sectional view of another exemplary embodiment of a wearable device for biofluid sensing.

FIG. 11B is a cross-sectional view of the device of FIG. 11A taken along line 11B-11B.

FIG. 11C is a cross-sectional view of the device of FIG. 11A taken along line 11C-11C.

### DEFINITIONS

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

As used herein, “biofluid” may mean any human biofluid, including, without limitation, sweat, interstitial fluid, blood, plasma, serum, tears, and saliva.

“Biofluid sensor” means any type of sensor that measures a state, presence, flow rate, solute concentration, solute presence, in absolute, relative, trending, or other ways in a biofluid. Biofluid sensors can include, for example, potentiometric, amperometric, impedance, optical, mechanical, antibody, peptide, aptamer, or other means known by those skilled in the art of sensing or biosensing.

“EAB sensor” means an electrochemical aptamer-based biosensor that is configured with a plurality of aptamer sensing elements that, in the presence of a target analyte in a fluid sample, produce a signal indicating analyte capture, and which signal can be added to the signals of other such sensing elements, so that a signal threshold may be reached that indicates the presence or concentration of the target analyte. Such sensors can be in the forms disclosed in U.S. Pat. Nos. 7,803,542 and 8,003,374 (the “Multi-capture Aptamer Sensor” (MCAS)), or in U.S. Provisional Application No. 62/523,835 (the “Docked Aptamer Sensor” (DAS)).

“Continuous monitoring” means the capability of a device to provide at least one measurement of biofluid determined by a continuous or multiple collection and sensing of that measurement or to provide a plurality of measurements of biofluid over time.

“Chronological assurance” means the sampling rate or sampling interval that assures measurement(s) of analytes in biofluid in terms of the rate at which measurements can be made of new biofluid analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5- to 30-minute lag time between analytes in blood emerging in interstitial fluid), but the resulting sampling interval is independent of lag time, and furthermore, this lag time is inside the body, and therefore, for chronological assurance as defined above and interpreted herein, this lag time does not apply.

As used herein, “determined” may encompass more specific meanings including but not limited to: something that is predetermined before use of a device; something that is determined during use of a device; something that could be a combination of determinations made before and during use of a device.

As used herein, “sweat sampling rate” is the effective rate at which new sweat, or sweat solutes, originating from the sweat gland or from skin or tissue, reaches a sensor that measures a property of sweat or its solutes. Sweat sampling rate, in some cases, can be far more complex than just sweat generation rate. Sweat sampling rate directly determines, or is a contributing factor in determining the chronological assurance. Times and rates are inversely proportional (rates having at least partial units of 1/seconds), therefore a short or small time required to refill a sweat volume can also be said to have a fast or high sweat sampling rate. The inverse of sweat sampling rate (1/s) could also be interpreted as a “sweat sampling interval(s)”. Sweat sampling rates or intervals are not necessarily regular, discrete, periodic, discontinuous, or subject to other limitations. Like chronological assurance, sweat sampling rate may also include a determination of the effect of potential contamination with previously generated sweat, previously generated solutes, other fluid, or other measurement contamination sources for the measurement(s). Sweat sampling rate can also be in whole or in part determined from solute generation, transport, advective transport of fluid, diffusion transport of solutes, or other factors that will impact the rate at which new sweat or sweat solutes reach a sensor and/or are altered by older sweat or solutes or other contamination sources. Sensor response times may also affect sampling rate.

As used herein, “sweat stimulation” is the direct or indirect causing of sweat generation by any external stimulus, the external stimulus being applied for the purpose of stimulating sweat. One example of sweat stimulation is the administration of a sweat stimulant such as pilocarpine. Going for a jog, which stimulates sweat, is only sweat stimulation if the subject jogging is jogging for the purpose of stimulating sweat.

Sudomotor axon reflex (SAR) is a biological response in which innervation of sweat glands occurs as a result of peripheral functionality of sudomotor units (i.e., the body will stimulate a group of sweat glands near the direct stimulation region).

As used herein, “sweat generation rate” is the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

As used herein, “measured” can imply an exact or precise quantitative measurement and can include broader meanings such as, for example, measuring a relative amount of change of something. Measured can also imply a binary measurement, such as ‘yes’ or ‘no’ type measurements.

As used herein, “sweat volume” is the fluidic volume in a space that can be defined multiple ways. Sweat volume may be the volume that exists between a sensor and the point of generation of sweat or a solute moving into or out of sweat from the body or from other sources. Sweat volume can include the volume that can be occupied by sweat between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

As used herein, “solute generation rate” is simply the rate at which solutes move from the body or other sources into sweat. “Solute sampling rate” includes the rate at which these solutes reach one or more sensors.

As used herein, “microfluidic components” are channels in polymer, textiles, paper, or other components known in the art of microfluidics for guiding movement of a fluid or at least partial containment of a fluid.

As used herein, “state void of sweat” is where a space or material or surface that can be wetted, filled, or partially filled by sweat is in a state where it is entirely or substantially (e.g., >50%) dry or void of sweat.

As used herein, “advective transport” is a transport mechanism of a substance or conserved property by a fluid due to the fluid's bulk motion.

As used herein, “diffusion” is the net movement of a substance from a region of high concentration to a region of low concentration. This is also referred to as the movement of a substance down a concentration gradient.

As used herein, “convection” is the concerted, collective movement of groups or aggregates of molecules within fluids and rheids, either through advection or through diffusion or a combination of both.

As used herein, a “volume-reduced pathway” is a sweat volume that has been reduced by addition of a material, device, layer, or other body-foreign substance, which therefore decreases the chronologically assured sweat sampling interval for a given sweat generation rate. This term can also be used interchangeably in some cases with a “reduced sweat pathway”, which is a pathway between eccrine sweat glands and sensors that is reduced in terms of volume or in terms of surfaces wetted by sweat along the pathway. Volume reduced pathways or reduced sweat pathways include those created by sealing the surface of skin, because skin can exchange water and solutes with sweat.

As used herein, “volume reducing component” means any component that reduces the sweat volume. In some cases, the volume reducing component is more than just a volume reducing material, because a volume reducing material by itself may not allow proper device function (e.g., the volume reducing material would need to be isolated from a sensor for which the volume reducing material could damage or degrade, and therefore the volume reducing component may comprise the volume reducing material and at least one additional material or layer to isolate volume reducing material from said sensors).

As used herein “pressure-permeated component” is a component that requires pressure to be permeated by sweat. Pressure-permeated components may also include all known one-way valves, which are opened by pressure, including those known by those skilled in the art of microfluidics. Sweat can be occluded using pressure. In one example, antiperspirants use pressure to stop sweat. Therefore, a pressure-permeated component can be designed to allow sweat flow at the low pressures that correlate with low sweat rates.

As used herein, a “horizontally-confining component” is a component that substantially prevents fluid from spreading horizontally along the skin surface.

As used herein, a “curable fluid or gel” is a fluid or gel that either dries or chemically cures into a solid.

As used herein, “hydrophobic” refers to materials through which non-charged non-hydrophilic solutes (hydrophobic solutes) will diffuse, but through which charged or hydrophilic solutes will not diffuse. For example, silicone-based oils or polymers can allow a hydrophobic analyte such as ethanol or such as cortisol or other steroid hormones to diffuse through them, but can block ions such Ca⁺, K⁺, Na⁺ and Cl⁻ and OH⁻ or H⁺ (e.g., block pH altering solutes). Quantitatively, we will use a testable definition for hydrophobic as being a material that has diffusive flux and/or solubility for solute ions such as Nat, Cl⁻, OH⁻ or H⁺ that is at least 100× lower than for solutes such as ethanol or cortisol. Simply, the hydrophobic solutes, even though larger in size, diffuse more rapidly through hydrophobic material. This same analogy can be applied in the present invention to gas molecules as well.

### DETAILED DESCRIPTION OF THE INVENTION

This specification builds upon on PCT/US15/32893, filed May 28, 2015, the disclosure of which is incorporated by reference herein in its entirety. The disclosed invention applies at least to any type of biofluid sensing device that measures sweat, sweat generation rate, sweat chronological assurance, its solutes, solutes that transfer into sweat from skin, a property of or things on the surface of skin, or properties or things beneath the skin. The disclosed invention applies to biofluid sensing devices which can take on forms including patches, bands, straps, portions of clothing, wearables, or any suitable mechanism that reliably brings sweat stimulating, sweat collecting, and/or sweat sensing technology into intimate proximity with sweat as it is generated. Some embodiments of the disclosed invention utilize adhesives to hold the device near the skin, but devices could also be held by other mechanisms that hold the device secure against the skin, such as a strap or embedding in a helmet.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

Certain embodiments of the disclosed invention show sensors as simple individual elements. It is understood that many sensors require two or more electrodes, reference electrodes, or additional supporting technology or features that are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: a sweat sensor; an impedance sensor; a sweat volume sensor; a sweat generation rate sensor; and a solute generation rate sensor. Certain embodiments of the disclosed invention show sub-components of what would be biofluid sensing devices with more sub-components needed for use of the device in various applications, which are obvious (such as a battery), and for purpose of brevity and focus on inventive aspects are not explicitly shown in the diagrams or described in the embodiments of the disclosed invention. As a further example, many embodiments of the disclosed invention could benefit from mechanical or other means known to those skilled in wearable devices, patches, bandages, and other technologies or materials affixed to skin, to keep the devices or sub-components of the skin firmly affixed to skin or with pressure favoring constant contact with skin or conformal contact with even ridges or grooves in skin, and are included within the spirit of the disclosed invention.

The invention includes reference to the article in press for publication in the journal IEEE Transactions on Biomedical Engineering, titled “Adhesive RFID Sensor Patch for Monitoring of Sweat Electrolytes”; the article published in the journal AIP Biomicrofluidics, 9 031301 (2015), titled “The Microfluidics of the Eccrine Sweat Gland, Including Biomarker Partitioning, Transport, and Biosensing Implications”; as well as PCT/US16/36038, which are incorporated herein by reference in their entirety. Techniques for concentrating a biofluid sample are disclosed in PCT/US16/58356, and U.S. Provisional Application No. 62/457,604, which are also hereby incorporated herein by reference in their entirety.

Sweat stimulation, or sweat activation, can be achieved by known methods. For example, sweat stimulation can be achieved by simple thermal stimulation, chemical heating pad, infrared light, by orally administering a drug, by intradermal injection of drugs such as methylcholine or pilocarpine, and by dermal introduction of such drugs using iontophoresis. A device for iontophoresis may, for example, provide direct current and use large lead electrodes lined with porous material, where the positive pole is dampened with 2% pilocarpine hydrochloride and the negative one with 0.9% NaCl solution. Sweat can also be controlled or created by asking the subject using the patch to enact or increase activities or conditions that cause them to sweat. These techniques may be referred to as active control of sweat generation rate.

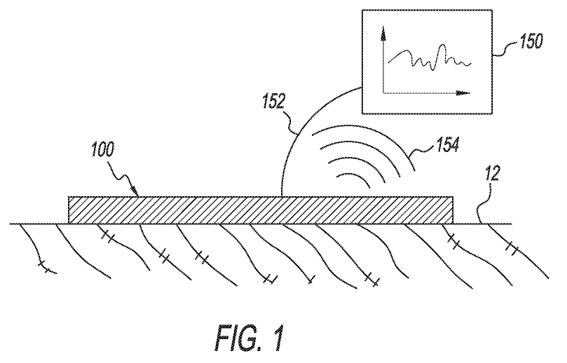

With reference to FIG. 1, a biofluid sensing device 100 is placed on or near skin 12. In an alternate embodiment, the biofluid sensing device may be fluidically connected to skin or regions near skin through microfluidics or other suitable techniques. The device 100 is in wired communication 152 or wireless communication 154 with a reader device 150. In one embodiment of the disclosed invention, the reader device 150 is a smart phone or portable electronic device. In alternate embodiments, device 100 and reader device 150 can be combined. In further alternate embodiments, communication 152 or 154 is not constant and could be a one-time or periodic data download from the device 100 once it has completed measurements of sweat.

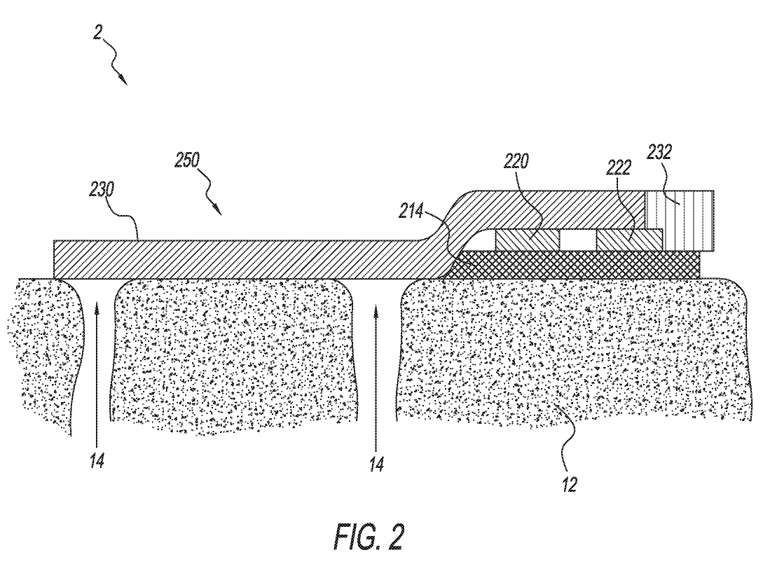

With reference to FIG. 2, a microfluidic component 230 carries sweat 14 from skin 12 to an analyte-specific primary sensor 220 that is placed on an impermeable substrate 214. The primary sensor 220 measures the presence, concentration, or other property of one or more solutes in sweat. For example, sensor 220 can be an impedance sensor for a cytokine biomarker, an ion-selective electrode to measure sodium, or an electrochemical aptamer-based (EAB) sensor to measure cortisol. One or more secondary sensors 222, such as a drift-free reference electrode, or a sensor to detect the presence of sweat, such as a galvanic skin response sensor, or a sensor to measure the flow rate of sweat, such as a micro-thermal flow rate sensor, or a temperature sensor, or other sensor may also be included. The impermeable substrate 214 can be a polyimide film. The microfluidic component 230 could be, for example, paper, a polymer microchannel, a tube, or a gel, or other means to transport sweat from skin to the sensors. The device is attached to skin by an adhesive (not shown), which may be a pressure sensitive, liquid, tacky hydrogel, which promotes robust electrical, fluidic, and iontophoretic contact with skin. For continuous monitoring, the microfluidic component 230 could wick sweat past the sensors 220, 222 to a hydrogel component 232, that continuously absorbs and pumps sweat from skin 12 and across the sensors at the rate at which sweat is generated from the skin. The device may be covered with a protective component (not shown), made of material such as one that is porous to sweat, one that wicks sweat like a hydrogel or textile, or one that is impermeable to sweat. This example is provided to show that the goals of the disclosed invention may be accomplished in multiple ways, and materials, elements and components of the disclosed invention can function in several configurations. Therefore, the specific example drawings provided should not be interpreted in a limiting manner.

Many biofluid sensing device applications place delicate sensors in dynamic environments for extended periods of time, which can expose the sensors to shear, abrasion, compression, or other forces through single or repeated contact with skin, or device components, such as wicking materials. Ionophore sensors and sensors that rely on a monolayer of a probe, such as impedance-based antibody or EAB sensors, are especially vulnerable to damage, which can introduce significant error into measurements of analytes that are present in sweat at very low concentrations (μM to pM and lower). Therefore, some sort of protection for the sensor may be required, and is provided by embodiments of the present disclosure.

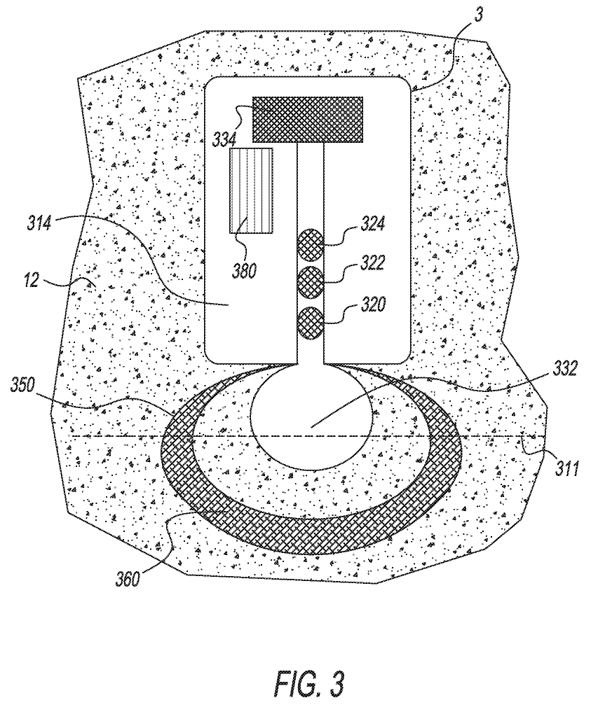

FIG. 3 presents a top view of at least a portion of the disclosed invention that protects sensors from damage, and in which like numerals refer to like features of previous figures (e.g., 320 is a sensor like sensor 220 of FIG. 2). Embodiments of the disclosed invention may resolve such challenges by having a sensor that can be protected from contact with skin by use of a protective material 332, for example. The device 3 has a sweat impermeable substrate 314 upon which a protective component 332 and electronics 380 are attached. The protective component 332 is in fluidic communication with the sensors 320, 322, 324, and with a pump material 334, and is configured to wick or transport sweat from the skin, across the sensors and to the pump. During use of the device, the skin 12 could move horizontally and abrade against the sensors 320, 322, 324. However, with the inclusion of the protective component 332, the sensors can be placed off the skin, and thereby protected from damage. A variety of materials can be used for the protective component 332, as long as the material is capable of adhesion to device surfaces and skin, and is capable of collecting a sweat sample and transporting the sweat to facilitate sensor function. Non-limiting examples include rayon, a textile, a paper microfluidic material, an aerogel, a low density gel, dialysis membrane material, a porous polymer, nafion, or an in-situ deposited or electro-deposited polymer that is porous and deposited onto the sensor.

Having provided solutions to the problem of sensor abrasion, embodiments of the disclosed invention also have reduced sweat volumes through the use of sensor-centered sweat flow, as disclosed in PCT/US15/32893. Sensor-centered flow involves directing new sweat from sweat glands toward the center of device sensors. To illustrate the advantage of having sensor-centered sweat flow, consider the case where the sweat sample flow is not centered on the sensor. When such a flow of sweat, e.g., one primarily centered to one side or adjacent to the sensor, reaches the sensor, the sensor will see non-uniform sweat flow, with relatively faster flow near where the sweat flow is targeted, and relatively slower flow elsewhere. Having slower sweat flow on part of the sensor will cause older sweat to be measured along with newer sweat, which increases the chronological sampling interval.

For embodiments using circular sensors, having the sweat flow centered on the sensor optimizes sweat sampling rate for a given sweat generation rate, providing sampling rates as much as ˜6× faster than a non-centered flow, as taught by Sonner, et al., in Biomicrofluidics. 2015 May 15; 9(3):031301. Doi: 10.1063/1.4921039. For embodiments using non-circular sensors, a centered flow would similarly improve sweat sampling rates.

While the theoretical benefits of configuring a biofluid sensing device with sensor-centered flow seem apparent, in practice, easily and reliably achieving alignment between device sensors and sweat glands poses a difficulty. Sweat glands are not uniformly distributed in skin, having variations in density between different body parts, and having random distribution in any one area of the body. Therefore, some embodiments of the disclosed invention are configured to allow sensors or other device components to self-align with sweat glands when placed on a device wearer's skin.

Other embodiments are configured to stimulate sweat while minimizing chemical contamination of the resulting sweat sample through use of sudomotor axon reflex (SAR) sweat stimulation, as disclosed in PCT/US2016/17726, which is incorporated herein by reference in its entirety. By using SAR sweat stimulation, the device can stimulate sweat glands within close proximity of a sensor array or sweat sample collector to generate sweat directly underneath the sensors or sweat collector. In combination, SAR sweat stimulation and sensor centered flow can greatly improve sweat sampling rates and reduce necessary sweat volumes, while decreasing contamination of the sweat sample.

With further reference to FIG. 3, the disclosed embodiment protects sensors from damage and implements sensor-centered flow and SAR sweat stimulation. Partially surrounding the protective material 332 is a ring-shaped region 350 containing a sweat stimulation compound 360 such as carbachol, methylcholine, acetylcholine, pilocarpine, or other suitable chemical. The ring shaped region 350 is placed at a distance from the protective material 332, for example between 1 and 5 mm, that optimizes SAR response by sweat glands located directly under the material 332. In some embodiments, the sweat stimulation compound may be introduced to the skin by iontophoresis. Preferentially, however, sweat stimulation will occur by passive diffusion into the skin, which may need to be facilitated by skin surfactants or chemical penetration enhancers as used in the art of transdermal drug delivery, for example by suspension in diols such as propylene glycol. See, Pathan, I., et al., “Chemical Penetration Enhancers for Transdermal Drug Delivery Systems,” Tropical Journal of Pharmaceutical Research, April 2009; 8 (2): 173-179.

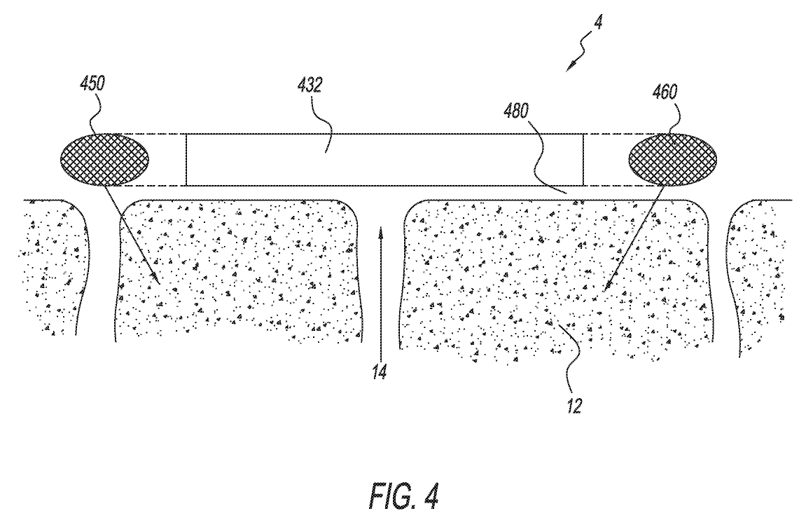

FIG. 4 is an alternate, cross-sectional view of the embodiment depicted in FIG. 3, as bisected along axis 311, and in which like numerals refer to like features of previous figures. The device 4 includes a protective component 432, and ring-shaped region 450 containing sweat stimulating compound 460. The device is placed on skin 12 over an eccrine sweat duct 14, and having a sweat volume 480 under the protective component 432. Sensor-centered flow can be facilitated by appropriately configuring the protective component 432 to directly and efficiently direct sweat across the sensors using known microfluidic techniques. For example, the skin-facing side of the protective component 432 may include a polymer with defined trenches to facilitate sweat flow, where the bottom of said trenches is the surface of a sweat impermeable membrane. Such trenches could have a geometry that promotes directional capillary flow, and therefore can be designed to move sweat toward the sensors. For example, if sweat wetted such a trench mid-way between the edge of the protective component and its radial center, then the sweat would wick to the radial center of the protective component 432, and from there to the sensors.

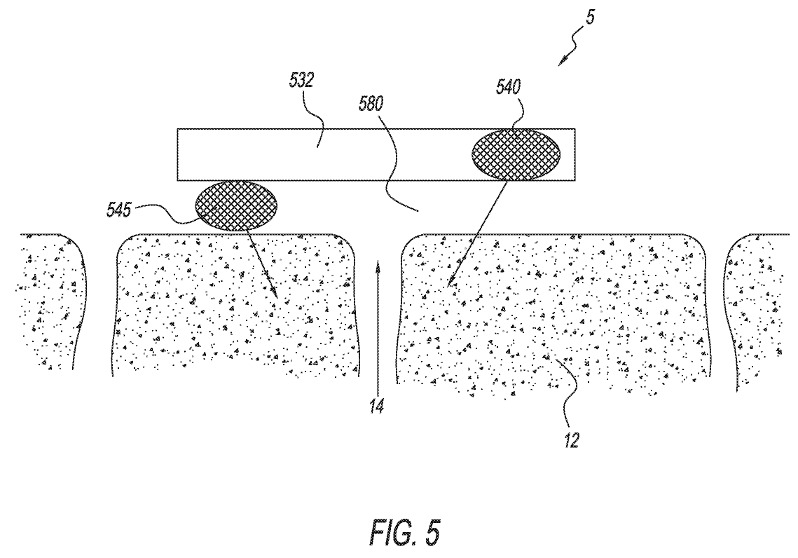

With reference to FIG. 5, where like numerals refer to like features of previous figures, in some embodiments, the device 5 may not include a ring shaped region, but instead would include a sweat stimulating compound inside 540, or on 545, the protective component 532. Other embodiments (not shown) may include both a ring-shaped region, and stimulating compound incorporated in the protective component. Co-locating the stimulating compound with the protective component as depicted may increase the likelihood of contamination of sweat samples by the stimulating compound. Therefore, the stimulating compound locations may need to be patterned on, or in, the protective component 532 to reduce contamination, or the component may include microfluidic channels, barriers, or track-etched membranes (not shown) to prevent or reduce contamination. As in the previous example, the protective component 532 may include a polymer on its skin-facing surface having defined trenches to facilitate sweat flow. However, in this embodiment, the trenches move uncontaminated sweat toward the sensors and move chemically-contaminated sweat away from the sensors. For example, if sweat wetted a trench mid-way between the edge of the protective component and its radial center, then the sweat would still wick to the radial, but if sweat wetted the trench closer to the edge, the trench would wick the sweat away from the radial center, and out of the device.

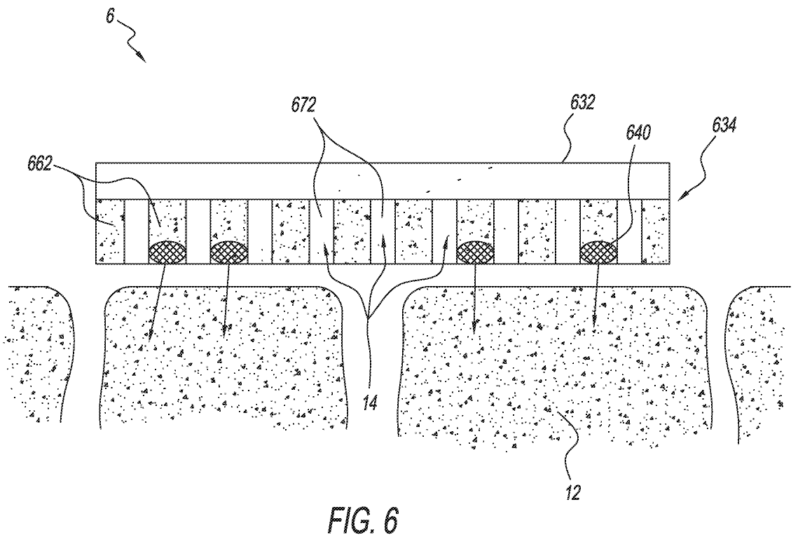

With reference to FIG. 6, where like numerals refer to like features of previous figures, a device 6 further includes a wicking material 634 that is configured to reduce sweat volume. Wicking material 634 has blocking areas 662 that largely prevent the flow of sweat, and flow areas 672 that allow sweat flow. In a preferred embodiment, the blocking areas would be >90% of the available surface area of material 634, which would reduce effective sweat volume by >100×. The wicking material 634 could be a layer of very thin paper or printed nano-cellulose, impregnated with wax to create blocking areas, as known by those skilled in the art of paper microfluidics. In all embodiments, cellulose or nano-cellulose could be replaced with polymer or other microfibers that may have less non-specific analyte absorption or some other desirable property. Additionally, when the device is placed on the skin, some flow areas will be aligned over sweat ducts 14, while others are not. The flow areas placed over a sweat duct would form a volume-reduced pathway for sweat. Likewise, flow areas not over a sweat duct and blocking areas would not be a part of the volume-reduced pathway. Therefore, the device will also self-align with sweat ducts to provide a sweat flow that is centered on the sensor.

Within a plurality of blocking areas 662 of the wicking material 634, some embodiments can be configured with a sweat stimulating compound 640, such as carbachol, acetylcholine, or methylcholine. The compound 640 may be arranged in different patterns to optimize sweat stimulation and minimize contamination of the sweat sample for various applications. Within the blocking areas, the compound would be separated from the skin by a sweat-dissolvable barrier, such as a material that dissolves in the presence of low pH solutions. In some embodiments, the compound may be co-formulated or mixed with an agent facilitating time release of the compound. Such time-release agents and techniques could be, for example, slow-release binders such as biocompatible polymers and copolymers, carrier agents that slow release, or agents that delay absorption of the stimulating compound, all as known in the art of sustained release chemistry. When activated, the sweat stimulating compound would diffuse into skin 12 slowly over time, for example over a 24-hour period. As with other embodiments, glycol, iontophoresis, or other means may be required to facilitate sweat stimulation. In this manner, the disclosed invention can supply low levels of prolonged sweat stimulation to facilitate continuous measurement of sweat analytes with minimal irritation to the device wearer, and with controlled sweat generation rates.

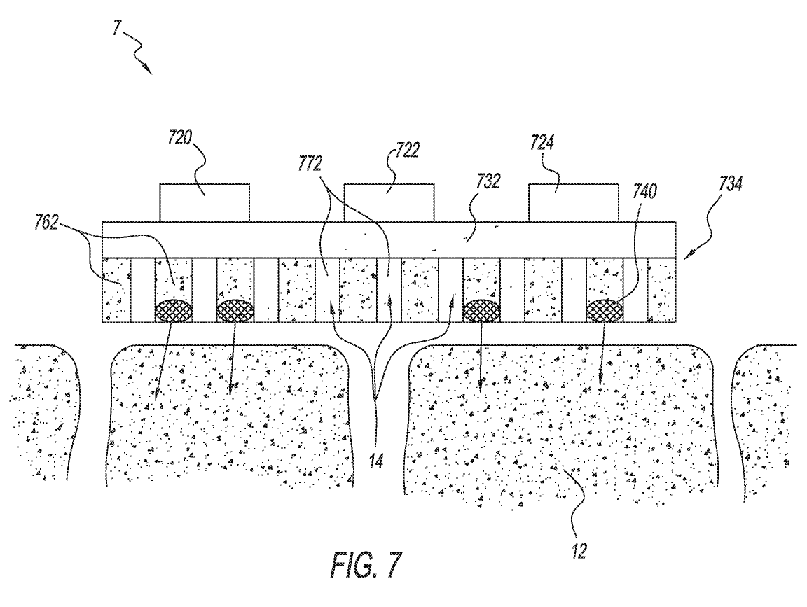

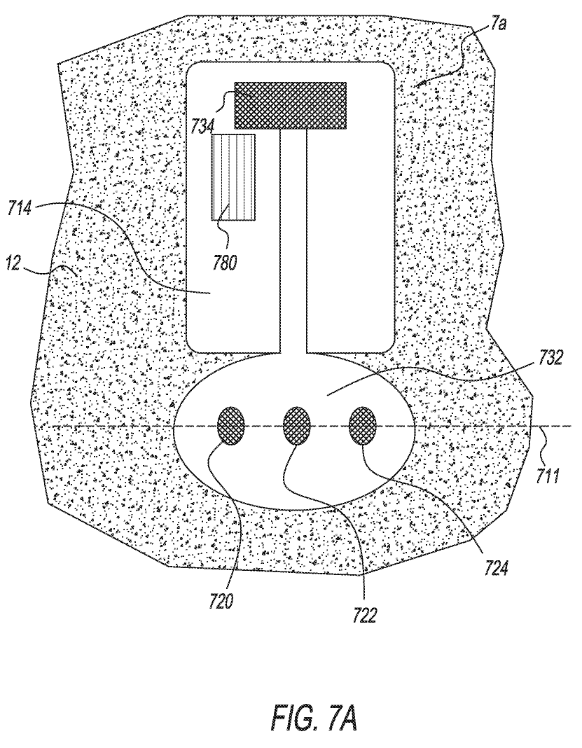

With reference to FIG. 7, in another embodiment of the disclosed invention, the device 7 includes sensors 720, 722, 724, configured on the protective material 732. This configuration protects the sensors from damage due to skin contact, and allows for a lower chronologically-assured sweat sampling interval by reducing overall sweat volume. FIG. 7A is a top view of the same embodiment with the depicted axis 711 illustrating the location of the cross-sectional view in FIG. 7.

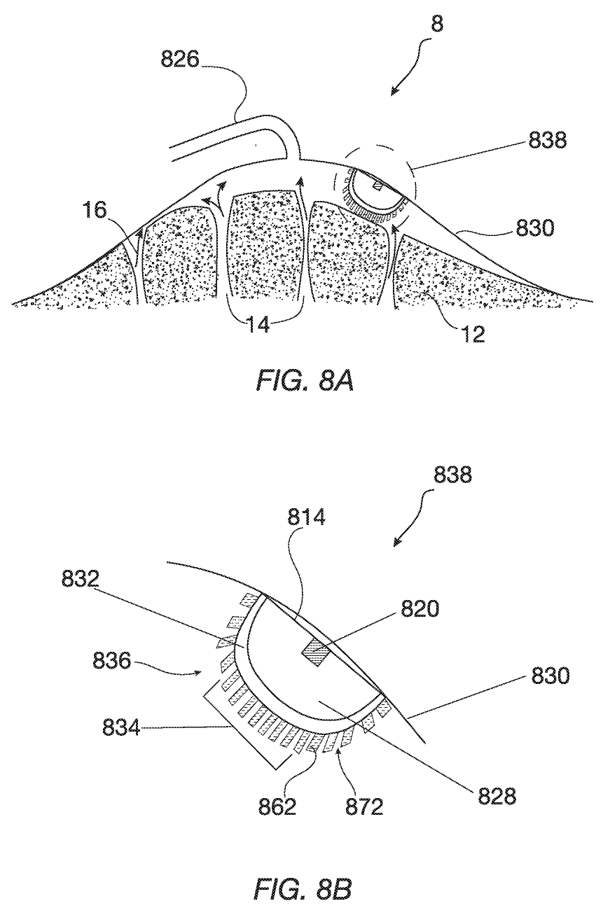

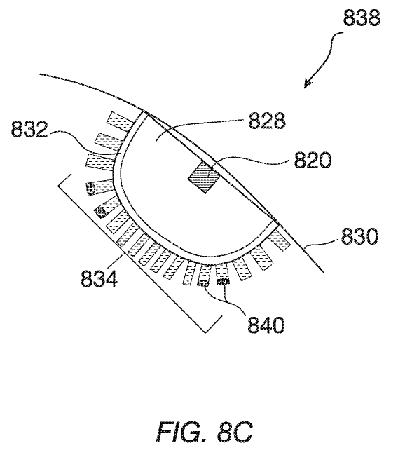

With reference to FIGS. 8A and 8B, in another embodiment of the disclosed invention, a biofluid sensing device 8 includes a concave sweat collector 830. During sweat sensing, the sweat collector 830 is sealed along an outer edge to a section of skin 12. Sweat enters the collector 830 from one or more sweat ducts 14, as indicated by the arrows 16. An evacuation channel 826 can be located on the opposite side of the collector 830 from sweat ducts 14 to enable a continuous flow of sweat through the collector, and/or to transport a sweat sample to additional sensors or other device components. One or more sensors (not shown) may be located in the evacuation channel 826 for measuring sweat attributes including, for example, concentrations of solutes, sweat rate, pH or salinity. As sweat flows through the collector 830, the sweat is sampled by a membrane-enhanced sensor 838. With reference to FIG. 8B, the membrane-enhanced sensor 838 includes a substrate 814, one or more analyte-specific sensors 820 for measuring the presence, concentration, or other property of one or more solutes or target analytes in the sweat sample, a sensor solution 828, and a protective component 836 at least partially surrounding the sensor. In the embodiment depicted in FIG. 8A, the membrane-enhanced sensor is located inside of the sweat collector 830, with the substrate 814 attached to the inner wall of the collector. In other embodiments, the membrane enhanced sensor is embedded in the wall of the collector, so that only a portion of the membrane-enhanced sensor 838 is exposed to the sweat sample.

Sensor 820 can be, for example, an impedance sensor for a cytokine biomarker, an ion-selective electrode to measure sodium, or an EAB sensor to measure cortisol, a drug, or another biomarker. Sensor solution 828 can be a neutral solution that is able to support diffusion of the one or more target analytes from at least the protective component 836 to the sensor 820, and support the reliable operation of the sensor. The salinity, pH, and other aspects of the solution 828 can be regulated to obtain ideal performance of the sensor 820. In some examples, the solution 828 includes a solvent as well as, optionally, one or more solutes. The solvent may be water, a glycol, an alcohol, an ionic liquid, an oil, or any other suitable liquid or fluid. The solvent may contain solutes. In an exemplary embodiment, an electrochemical aptamer-based sensor is stabilized in a pH 7, 50 μM NaCl solution. The thickness of the sensor solution 828, i.e., the distance from the protective component to the sensor, will influence operation of the device. The greater the thickness of the sensor solution 828, the longer the diffusive pathway from the protective component 836 to the sensor 820, and the larger the volume that must be equilibrated with the analyte concentration in the sweat sample. Generally, the thickness of the sensor solution 828 should be at least one of <3 mm, <1 mm, <300 μm, <100μ, <30 μm, <10 μm.

As shown in greater detail in FIG. 8B, protective component 836 comprises at least two membranes 832 and 834 that each at least partially surround the sensor 820. The first membrane 832 is hydrophobic and permiselective to a target analyte, allowing diffusion of the target analyte from the sweat sample to the sensor solution 828 in contact with the sensor 820. The hydrophobicity of the first membrane 832 also limits the reverse diffusion of the sensor solution 828, to maintain the solution in contact with the sensor 820. First membrane 832 can be, for example, a non-charged, hydrophobic silicone polymer, a hydrocarbon oil fluid membrane that could be highly permeable to an analyte such as ethanol, or other semi-permeable membrane material impregnated with an alkane or silicone oil or other hydrophobic liquid or fluid. First membrane 832 may also be selected to prevent diffusion of many, if not all, analytes that could interfere with sensor 820 and cause a false reading for the analyte of choice, such as acetaminophen, and thereby improve the selectivity, sensitivity, limit of detection, or combinations thereof, of the one or more sensors 820. First membrane 832 is adequately rigid or supported by a rigid material, such as a stainless steel mesh, or alternatively, is supported by the fluid pressure of the solution 828 and the second membrane 834. The first membrane 832 protects the sensor 820 from abrasion due to skin contact, while allowing hydrophobic or lipophilic molecules to diffuse through to the sensor.

Second membrane 834 substantially surrounds the first membrane 832 and can facilitate transport of a sweat sample to the first membrane. Second membrane 834 can also provide a reverse barrier between the first membrane 832 and the skin 12. In particular, second membrane 834 can be a hydrophilic track-etched membrane having a plurality of blocking areas 862 that largely prevent the flow of sweat, and a plurality of flow areas 872 that facilitate sweat flow. The flow areas 872 may include a hydrophilic solvent, a hydrogel, or other similar type of material which allows a sweat sample to move through the membrane. Alternatively, the flow areas 872 could comprise areas void of material located between each of the blocking areas, with the void forming a physical barrier between the skin and the first membrane 832. When the first membrane layer 832 comprises an oil membrane, the blocking and flow areas 862, 872 form a reverse barrier to prevent oil from permeating the second membrane and contacting the skin 12. In addition to the first and second membrane layers 832, 834, supplementary membranes could be provided that are comprised of materials with differing properties. For example, membranes could all be permeable to the target analyte but one is impermeable to salt and another is impermeable to pH, so that in combination they better block pH and salt but pass more easily the target analyte.

The thickness of protective component 836 will influence operation of the biofluid sensing device. If the thickness of the protective component is too great, the component can behave as a large sink or storage vessel for an analyte, and increase the diffusive pathway that the analyte must traverse to reach the sensor 820. Therefore, the thickness of the protective component may be at least one of <1 mm, <100 μm, <10 μm, <1 μm, or <0.1 μm.

As shown in FIG. 8C, in some embodiments the second membrane of the enhanced-membrane sensor can be configured with a sweat stimulating compound 840 such as, for example, carbachol, acetylcholine, pilocarpine, or methylcholine. The compound 840 may be arranged in different patterns on or within the membrane to optimize sweat stimulation and minimize contamination of the sweat sample. In this exemplary embodiment, membrane-enhanced sensor 838 is located inside of the collector 830 to sample as much of the collected sweat as possible without occluding or otherwise interfering with sweat flow from the skin.

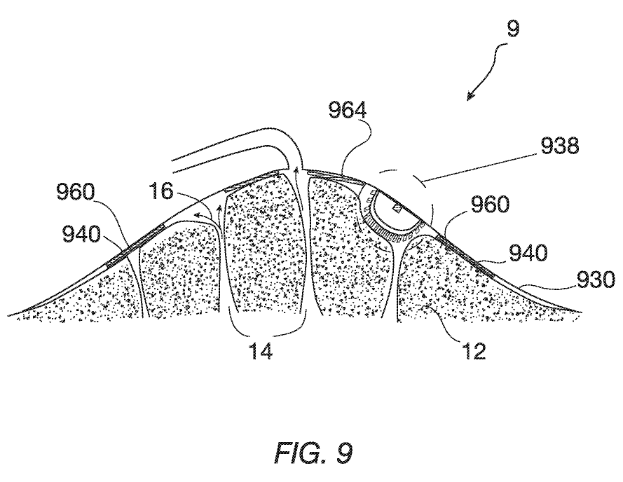

Referring now to FIG. 9, a biofluid sensing device 9 is shown having a collector 930 and a membrane-enhanced sensor 938 similar to that described in the text for FIGS. 8A-8C. In biofluid sensing device 9, a pair of continuous electrode rings 960, 964 are included inside of the collector 930 for stimulating sweat. Both of the stimulation electrode rings are in contact with skin 12. A sweat stimulating agent, e.g., carbachol, indicated at 940, is provided on one or both of the electrodes 960, 964 for delivery into the skin through iontophoresis. The electrodes 960, 964 may be activated periodically to deliver multiple, short doses of a sweat stimulant directly to the skin. Alternatively, the electrodes may be activated to deliver an initial, short dose of stimulant to the skin, followed by a delay to allow sweating to begin before subsequent doses of stimulant are administered, or any other stimulation protocol that provides sweat stimulation while minimizing irritation or discomfort at the skin surface.

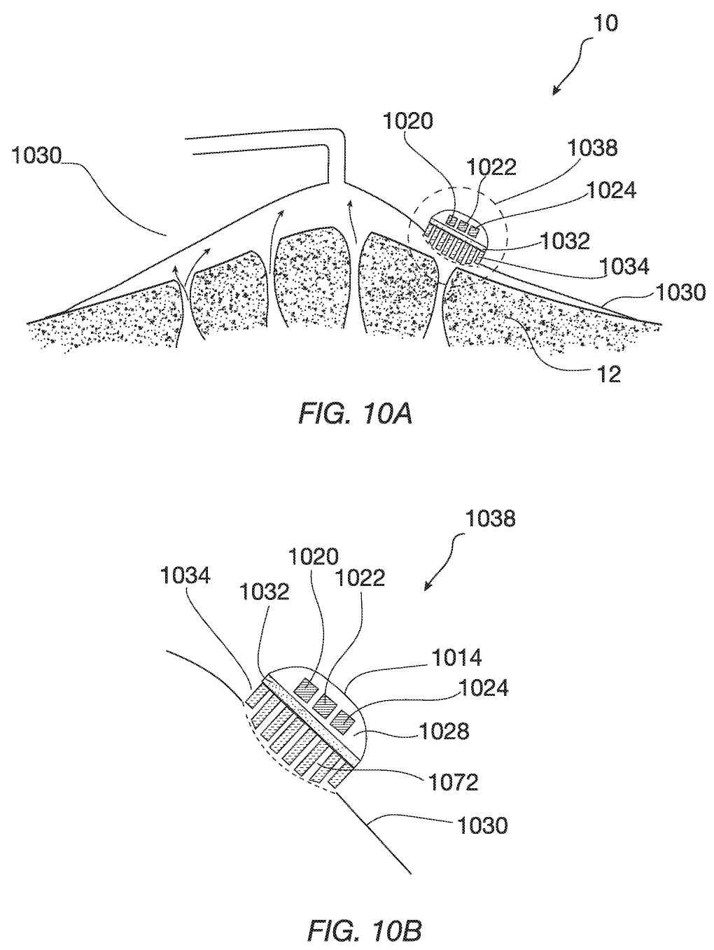

Referring now to FIGS. 10A and 10B, a biofluid sensing device 10 is shown with a membrane-enhanced sensor 1038 located outside of a collector 1030. In this embodiment, the membrane-enhanced sensor 1038 is depicted with three analyte-specific sensors 1020, 1022, 1024. These analyte-specific sensors can be selected to detect and measure the same target analyte in a sweat sample or, alternatively, one or more of the sensors may measure different analytes in the sweat sample. Where the sensors detect different analytes, the first membrane 1032 would allow diffusion of multiple different analytes. The sensors 1020, 1022, 1024 are maintained in a stabilizing solution 1028, between a substrate 1014 and a protective component, to optimize performance of the sensor. In this embodiment, the membrane-enhanced sensor 1038 is located at least partially outside of the collector 1030. A second membrane 1034 can extend at least partially through the surface of the collector 1030, and include one or more flow paths 1072 to convey a sweat sample from the interior of the collector to a first membrane 1032 and the sensors 1020, 1022, 1024. Selected hydrophobic molecules can diffuse from the flow paths 1072, through the first membrane 1032, and into the solution 1028. The concentration of each of the target analytes in the solution 1028 can be measured by the respective sensor. The biofluid sensing device 10 can also provide sweat stimulation via iontophoresis using a pair of electrodes, or through sweat stimulating components in the second membrane, as described in the previous embodiments.

In each of the embodiments shown in FIGS. 8A-10B, an analyte-specific sensor is housed between a substrate and membrane layers, and surrounded by a stabilizing solution. The membrane layers and stabilizing solution protect the sensor from abrasive contact with the skin 12. Additionally, the combination of a hydrophobic, oil-based inner membrane and a hydrophilic outer membrane enables a target analyte such as, for example, a drug, to be selectively diffused from a sweat sample, through the inner membrane, to an analyte sensor, while preventing the oil membrane from contacting the skin. The hydrophobic membrane layer also prevents hydrophilic species in the sample, such as pH, salt, or redox active molecules, from reaching the sensor. The multiple membrane layers as described herein also prevent a sensor stabilizing solution from diffusing away from the sensor, improving the stability and durability of the sensing device. It is envisioned that membrane-enhanced sensors as described herein may have other configurations, sizes, and arrangements. It should be understood that the depiction in the figures is not intended to be to scale, and the relative sizes and locations of the elements in the biofluid sensing devices can vary from what is depicted in the figures without departing from the scope of the invention.

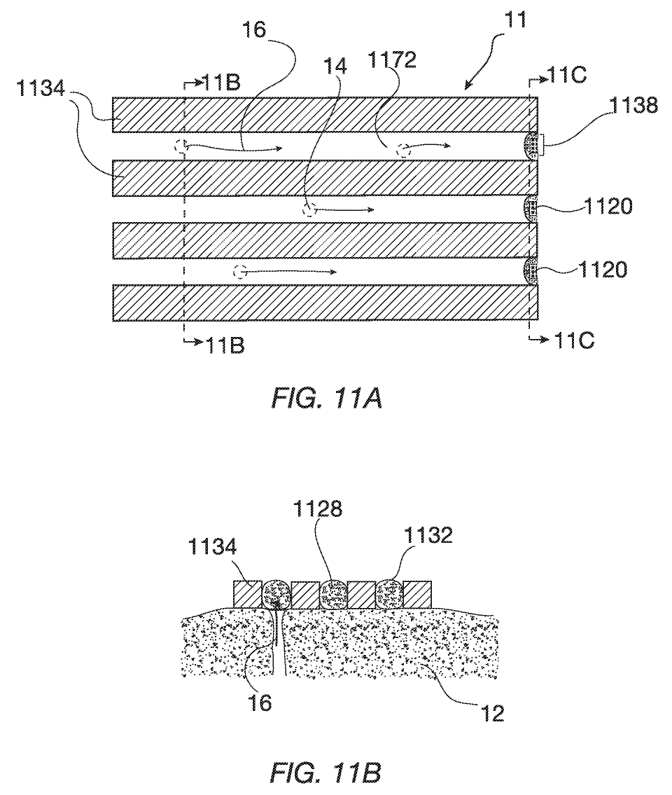

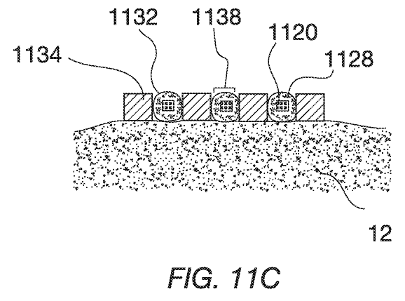

In another exemplary embodiment, shown in FIGS. 11A-11C, a biofluid sensing device 11, shown in FIG. 11A as a top-down view, includes a track-etched membrane 1134 having a plurality of flow channels 1172 for conveying a sweat sample. Sweat enters the flow channels 1172 from one or more sweat ducts 14, and flows in the direction of the arrows 16. A membrane-enhanced sensor 1138 is provided in one or more of the flow channels 1172, depicted here at the end of the channel. A wicking material, or other fluid conveying medium (not shown), can be placed in one or more of the channels 1172 for transporting sweat in the direction of the sensor. At the membrane-enhanced sensor 1138, a target analyte can diffuse through a hydrophobic, selectively permeable barrier 1132 into a neutral sensor stabilizing solution 1128. An analyte sensor 1120, specific to the target analyte, measures the concentration of the target analyte in the solution and provides a signal indicative of the concentration when the sensor is interrogated. In this embodiment, the track-etched membrane 1134 provides both a sweat path to one or more membrane-enhanced sensors 1120, and a protective component for preventing the skin from abrading the sensors.

This has been a description of the disclosed invention along with a preferred method of practicing the disclosed invention, however the invention itself should only be defined by the appended claims.

What is claimed is:

1. A biofluid sensing device comprising:
a sweat collector in contact with a skin surface; and
a membrane-enhanced sensor for measuring at least one of the presence and concentration of a target analyte in a sweat sample in the sweat collector.

2. The biofluid sensing device of claim 1, wherein the sweat collector has a concave configuration.

3. The biofluid sensing device of claim 1, wherein the membrane-enhanced sensor further comprises an analyte sensor specific to the target analyte and a protective component between the analyte sensor and the skin surface, the protective component including at least one hydrophobic barrier separating the analyte sensor from the sweat sample in the collector.

4. The biofluid sensing device of claim 3, wherein the protective component prevents contact between the analyte sensor and the skin surface.

5. The biofluid sensing device of claim 3, wherein the membrane-enhanced sensor further comprises a first membrane layer, a second membrane layer, and at least one analyte sensor specific to the target analyte.

6. The biofluid sensing device of claim 5, wherein at least one of the first and second membrane layers is selectively permeable to the target analyte.

7. The biofluid sensing device of claim 5, wherein the membrane-enhanced sensor further comprises a solution at least partially surrounding the analyte sensor, and wherein at least one of the salinity and pH of the solution is regulated to optimize performance of the analyte sensor.

8. The biofluid sensing device of claim 2, wherein the membrane-enhanced sensor is located inside of the collector.

9. The biofluid sensing device of claim 2, wherein the membrane-enhanced sensor is located at least partially outside of the collector, and includes at least one flow path extending through the surface of the collector to convey a sweat sample to the sensor.

10. The biofluid sensing device of claim 7, wherein the first membrane layer further comprises a hydrophobic barrier that diffuses the target analyte from the sweat sample to the solution, and the second membrane layer further comprises a hydrophilic track-etched membrane that conveys a sweat sample to the first membrane layer, the first and second membrane layers separating the skin from the analyte-specific sensor.

11. The biofluid sensing device of claim 10, wherein the second membrane layer prevents contact between the oil membrane and the skin.

12. The biofluid sensing device of claim 1, wherein the device further comprises a sweat stimulating component.

13. The biofluid sensing device of claim 11, wherein the sweat stimulating component further comprises at least two electrodes inside the sweat collector for stimulating sweat through iontophoresis.

14. The biofluid sensing device of claim 11, wherein the sweat stimulating component further comprises a chemical sweat stimulating compound arranged on or within the membrane-enhanced sensor.

15. A biofluid sensing device comprising:
a track-etched membrane having at least one flow channel for receiving a sweat sample from an eccrine sweat gland; and
at least one membrane-enhanced sensor in fluid communication with the at least one flow channel, the membrane-enhanced sensor including an analyte-specific sensor measuring at least one of the presence and concentration of a target analyte in the sweat sample.
